(19) United States
(12) Patent Application Publication
(10) Pub. No.: US 2023/0190135 A1
(43) Pub. Date: Jun. 22, 2023

(54) METHOD AND SYSTEM FOR USING TOOL WIDTH DATA TO ESTIMATE MEASUREMENTS IN A SURGICAL SITE

(71) Applicant: Asensus Surgical US, Inc., Durham, NC (US)

(72) Inventors: Tal Nir, Haifa (IL); Lior Alpert, Haifa (IL)

(21) Appl. No.: 17/955,486

(22) Filed: Sep. 28, 2022

Related U.S. Application Data
(60) Provisional application No. 63/249,373, filed on Sep. 28, 2021.

(51) Int. Cl. A61B 5/107; G16H 30/20; G06T 7/50

(52) U.S. Cl. CPC A61B 5/1076; G16H 30/20; G06T 7/50; G06T 2207/10021

(57) ABSTRACT

A system and method apply computer vision to images captured of a surgical site in order to measure the distance between surgical instruments at the surgical site. Images of the site are captured and analyzed to estimate a width in pixels of a first surgical instrument positioned at the treatment site. The depth coordinate Z of the tip of the first surgical instrument is determined by comparing the estimated tool width to the known physical width of the first surgical instrument, and the corresponding X and Y coordinates are estimated based on the Z coordinate. The coordinates of the tip of a second instrument are determined in the same manner, and the distance between the tips is then calculated, and the distance value is displayed as an overlay on the image display.

This application claims the benefit of U.S. Provisional Application No. 63/249,373, filed Sep. 28, 2021.

### BACKGROUND

Acquiring measurement data from a surgical site can be useful to a surgeon or other practitioner.

Size measurements within the surgical field are typically estimated by the user as s/he views the display of endoscopic images captured of the surgical site, and s/he may refer to other elements within the image to provide size cues (e.g., known diameters or feature lengths on surgical instruments) that facilitate estimation. In more complex cases, a sterile, flexible measuring “tape” may be rolled up, inserted through a trocar, unrolled in the surgical field, and manipulated using the laparoscopic instruments to make the necessary measurements.

Co-pending and commonly owned U.S. application Ser. No. 17/035,534, entitled “Method and System for Providing Real Time Surgical Site Measurements” describes a system and method that use image processing of the endoscopic view to determine sizing and measurement information for a hernia defect or other area of interest within a surgical site.

Co-pending and commonly owned U.S. application Ser. No. 17/099,761, entitled “Method and System for Providing Surgical Site Measurements” describes a system and method that use image processing of images of the endoscopic view to estimate or determine distance measurements between identified measurement points at the treatment site. The measurements may be straight line point to point measurements, or measurements that follow the 3D topography of the tissue positioned between the measurement points.

The above-referenced applications are incorporated herein by reference, and features and methods that they described may be combined with the concepts described in this application.

This application describes a new system and method for providing distance measurements between measurement points at a treatment site.

### BRIEF DESCRIPTION OF THE DRAWINGS

FIG. 1A is a block diagram schematically illustrating a system according to the disclosed embodiments.

FIG. 1B is a perspective view of a distal part of a robotic manipulator arm, and a surgical instrument to be mounted to the distal part of the robotic manipulator arm.

FIG. 2 is a schematic diagram generally depicting the described method for estimating measurements.

FIG. 3 is a schematic diagram depicting the method for estimating the 3D coordinates of surgical instruments positioned at the surgical site.

FIG. 4 shows an example of a graphical user interface (GUI) displaying an image of a surgical site, in which two surgical instruments are positioned. Overlays shown on the GUI display depict measurement points at the instrument tips (each marked by an overlay symbol in the form of a small circle), and measurement data representing the distance between the pair of points.

FIG. 5 schematically illustrates data derived from the image data corresponding to the lateral and distal extents of two surgical instruments, and the instrument tips between which measurements are estimated.

FIG. 6A shows a GUI displaying an image of a surgical site in which two surgical instruments are positioned. An overlay depicts a measurement point offset from the distal tip of one of the surgical instruments.

FIG. 6B is similar to FIG. 6A, but shows a digital pin or tag displayed on the display at the location where the measurement point is located.

FIG. 7 shows one example of a robotic surgical system that may be used in conjunction with the measurement concepts described in this application.

### DETAILED DESCRIPTION

This application describes a system and method for use in conjunction with surgical instruments that are used to perform diagnostic or therapeutic tasks at a surgical site. The system and method analyze the surgical site using computer vision and measure a distance between surgical instruments within the surgical site. The described system and method may be used in conjunction with a surgical robotic system in which the instruments and/or camera are maneuvered by robotic manipulators. They might also be used in non-robotic procedures, where the user maneuvers hand-held instruments within the body.

System

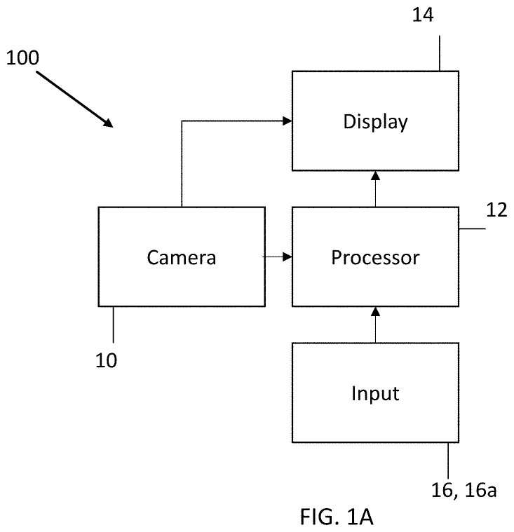

Referring to FIG. 1A, an exemplary system 100 preferably includes a camera 10, one or more processors 12, a display 14 that displays images captured by the camera, and an input device 16. The camera 10 may comprise any type of camera suitable for capturing images within a body cavity, including stereoscopic or monocular endoscopic cameras. The camera 10 has a known f parameter (ratio of pixels to angle in radians).

The display 14 may be any form of image display suitable for displaying images from the camera, including a monitor, tablet, touch screen display, heads up display, head-worn display, etc. Where the system is used with a robotic assisted surgical system, the display may be a screen positioned at or near the surgeon console at which the surgeon gives input used by the robotic surgical system to direct movement of the surgical instruments at the surgical site.

The input device 16 is used to identify the type(s) or characteristic(s) of surgical instruments that will be used at the surgical site to, or by, the system. The term “input device” is used for convenience, but it should be understood that it can be an input device, memory device, or other feature or combination of features that ensures that the processor(s) have the relevant instrument characteristics needed to be used for the measurement estimations. Several non-limiting examples of input devices are given in the next few paragraphs. As will be understood from the method described in the “Method” section of this application, the instrument characteristic used by the one or more processors to estimate distances is the physical width of the surgical instrument in the lateral dimension. In preferred embodiments, this is typically the diameter of the surgical instrument main shaft rather than the width of the instrument's tip, which may be narrower or wider than the main shaft In alternative embodiments, the width of a particular portion of the tip may be used.

Referring to FIG. 1B, if the system 100 is one used with a robotic surgical system for which the surgical instrument(s) 30 are mounted to robotic arm 32, the surgical instrument (or an adapter used to mount the surgical instrument to the robotic arm) may be equipped to actively or passively communicate information regarding the instrument types to the system. For example, each surgical instrument 30 may include an integrated circuit having a memory in which data about the surgical instrument is stored. The system is configured to receive or retrieve the data from the memory of the integrated circuit. This may be via contactless transmission or receipt of the data to or by a reader, such as by using RFID communication. In alternative embodiments, the integrated circuit may be one from which the system receives/retrieves the data by other means, such as an EEPROM device having memory that is read via an electrical connection to the EEPROM device.

In a preferred arrangement, the integrated circuit is part of an RFID tag 34 that also includes an antenna that communicates the data stored in the memory to a reader 36 on or in proximity to the robotic arm. Such technology is beneficial in that it allows data to be communicated through the surgical drape(s) 38 positioned between the surgical instrument 30 and robotic arm 32 so as to maintain the sterile field.

In alternative embodiments, a bar code, QR code or magnetic stripe/region may be positioned on each instrument to be read by a corresponding reader on the robotic arm etc. For other systems (including those used with manual surgical instruments and/or robotically manipulated surgical instruments), the RFID tag, bar code, QR code etc. may be read by a reader that is not positioned on a robotic arm. For example, the reader may be manually brought into proximity with the RFID tag, bar code, QR code etc. As yet another example, a part of the instrument, such as the tip, may be held in front of a camera, and the resulting images processed in accordance with an algorithm programmed to determine the instrument type based on a visual characteristic such as its color, markings (QR code or bar code markings, or other markings) or shape.

Various other techniques may instead be used to input the tool information to the system. For example, an eye tracking device, head tracking device, touchpad, trackpad, mouse (i.e., any form of user-moveable device that moves a cursor displayed on a display, whether a computer mouse or other device), touch screen or keyboard may be used to input the surgical instrument information, or to select it from a menu or other display of options. A microphone may be used to receive vocal input of the relevant information.

The data input by any of the described inputs may provide the surgical instrument width, or it might instead provide other identifying information from which the one or more processor(s) can retrieve the width from memory accessible by the processor(s). For example, the data might communicate an identifier such as a reference code for the instrument, or specify the type of instrument (e.g., Maryland dissector). In these embodiments, the width data is stored in the memory, and the data read or received from the instrument or input by the user is used by the system to retrieve the relevant width data from the memory. In yet other embodiments, the data read or received from the instrument or input by the user may be data from which the one or more processor(s) can mathematically derive the instrument width.

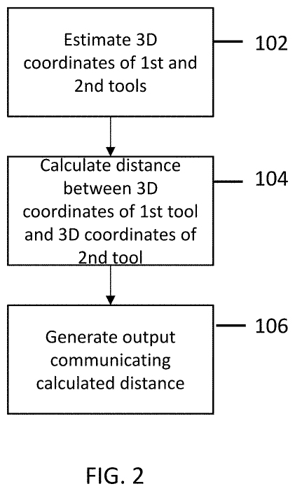

While the above discussion focuses on ensuring the one or more processor(s) make use of the actual surgical instrument width in the calculations used to estimate tool distances, the input 16 or other sources of data may also be used to communicate information that aids the processor(s) in carrying out the Method described below. For example, the processor(s) may have been trained, using machine learning or other methods, to recognize particular types of surgical instruments in images. Data pertaining to the types of surgical instruments can thus be used to aid the image processing analysis of the image data to identify the surgical instruments and their extents at the surgical site (Step 202 described with respect to FIG. 3). Data pertaining to the region where a surgical instrument is located within the surgical site can inform the processor(s) as to where to “look” on the images to locate each instrument. This latter form of data might include data indicating which of multiple robotic arms of a robotic system is carrying the surgical instrument and which of the arms is carrying the camera, or it might include data from an eye tracking system (such as that described in U.S. Pat. No. 9,360,934, which is incorporated by reference) identifying the area of the displayed image of the surgical field the user is looking at.

As depicted, the system is configured so that the one or more processors 12 receive the images from the camera 10 and data from the input 16. The processor(s) include(s) at least one memory storing instructions executable by the processor(s) to carry out the following steps, as well as others listed in the Method section:

(i) receive input corresponding to the width of the surgical instruments to be used in the surgical field,

(ii) receive image data corresponding to the images of the surgical field captured by the camera,

(iii) identify the surgical instruments in the image data,

(iv) identify the outlines of the surgical instruments in the image data, such as using edge detection and/or other segmentation techniques,

(v) using straight line approximation, estimate the width of each surgical instrument in the images;

(vi) for each instrument, by comparing the known width to the estimated width in the images, estimate the depth Z of the tip of the instrument; and

(vii) for each instrument, after estimating the depth of the instrument's tips, estimate the X and Y coordinates of the instrument tips to obtain the estimated 3D coordinates of the instrument tips;

(viii) estimate the distance between the tips of the instruments by calculating the Euclidean distance between their 3D coordinates; and

(ix) generate output communicating the measured distances to the user. The output may be in the form of graphical overlays on the image display displaying the measurement data (as described in connection with the drawings), and/or in other forms such as auditory output.

Method

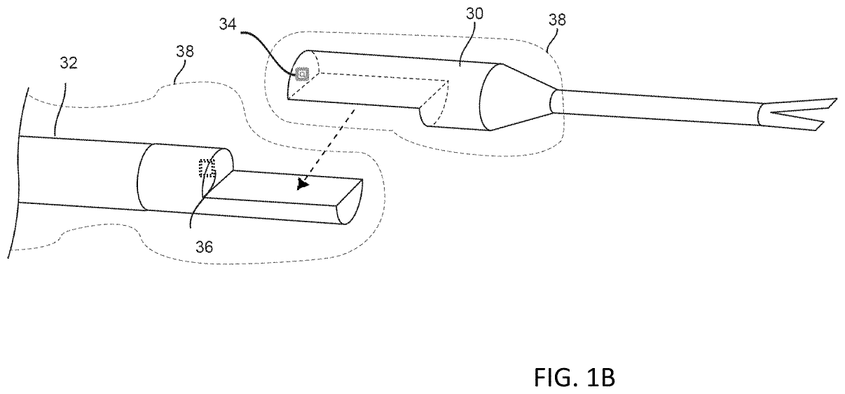

FIG. 2 generally depicts a method of estimating a distance between a point on a first surgical instrument and a point on a second surgical instrument. The examples given in this application will describe the method as estimating the distance between the tips of the first and second instruments, but the method may be modified to make distance estimations for distances between other points on the instruments.

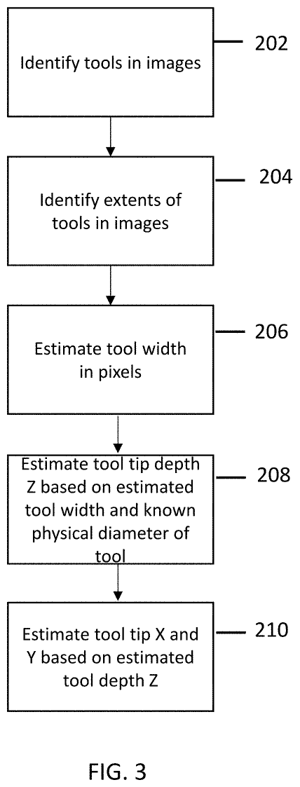

Referring to FIG. 2, in Step 102 the system estimates the 3D coordinates of the tip on each instrument from which the distance measurement is to be estimated. Then, the distance between the 3D location of the tips is estimated. Step 104. In Step 106, the estimated distance is communicated to the user. The form of communication may be generation and display of an overlay on the image display stating the estimated distance, as shown in FIG. 4. Graphical icons, such as the circles shown over the tool tips in FIG. 4, may be displayed to mark the points between which the measurement has been estimated. Note that the “tip” may not correspond to the actual physical tip of the surgical instrument. It may instead be a point corresponding to the distalmost extent of the surgical instrument and centered between the lateral extents of the surgical instrument, as depicted by the circles shown in FIG. 4. Further graphical icons, such as a line (not shown) extending between the points may also be displayed.

The block diagram of FIG. 3 provides greater detail of the method carried out in Step 104.

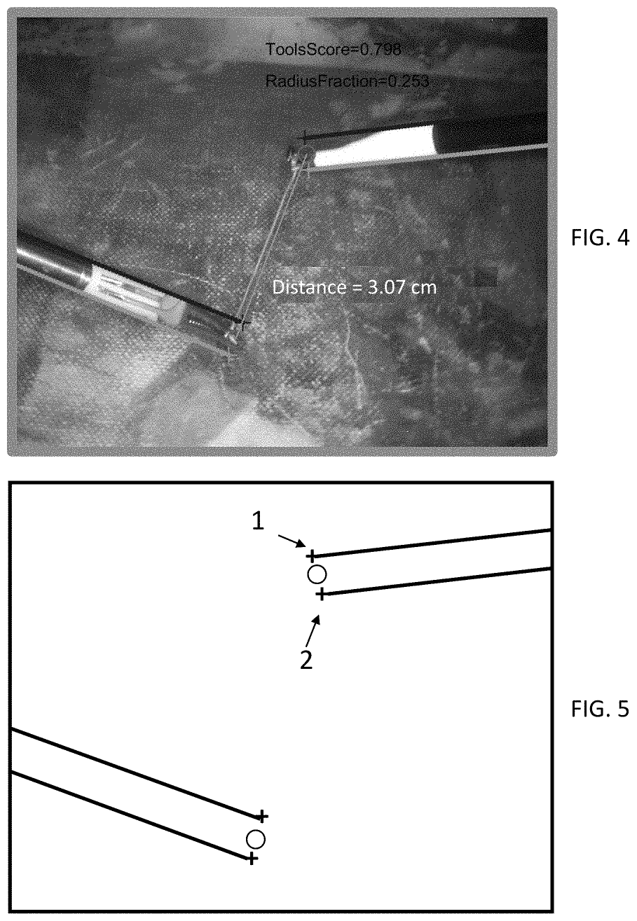

Surgical instruments with known diameters (e.g., whose diameters have been received or retrieved by the processors based on data communicated from an IC on an RFID tag as described above) are introduced into a surgical site in a body cavity. Images of the instruments in the surgical site are captured using the camera, which also is positioned in the body cavity. The processor analyzes the image data from the images and identifies the tools in the images. Step 202. The extents or outlines of the instruments are identified in the image, Step 204, using edge detection and/or related segmentation techniques, which are known to those skilled in the art. Referring to FIG. 4, the lines running longitudinally along the edges of the instruments mark the lateral-most boundaries of the instruments. The distalmost extents of the instruments are represented by the “+” icons at the distal ends of each of those lines. FIG. 5 is similar to FIG. 4 but shows only the lines and icons for ease of viewing.

Using straight line approximation, the width in pixels at the tool's tip is estimated in Step 206. This corresponds to the orthogonal distance between the lines marking the lateral-most boundaries of the instruments in FIG. 4. In Step 208, the width determined in Step 206 is compared to the known physical diameter in order to estimate the depth at the tip, or the Z coordinate of the tip.

The described method is highly beneficial in that it allows the Z coordinate to be determined even if the only known camera parameter is its f parameter. FIG. 5 identifies Point 1 and Point 2 as the distal and lateral extents of a surgical instrument in the image plane. Assuming a projective camera model with known f (ratio of pixels to angle in radians), the u,v coordinates in the image plane corresponding to the 3D world coordinates of Point 1 and Point 2 may be expressed as follows:

u1=f*X1/Z+px (1)

v1=f*Y1/Z+py (2)

u2=f*X2/Z+px (3)

v2=f*Y2/Z+py (4)

As depicted in FIG. 5, Points 1 and Points 2 are diametrically opposite points on the lines identifying the opposite edges of the image of the tool (i.e., which would, on the actual instrument, fall along a common circumference). In these equations:
Coordinates u1, v1 and u2, v2 are the coordinates of Points 1 and Points 2, respectively, in the image plane; and
Coordinates px, py are the coordinates of the principal point, the pixel coordinate of the intersection of the optical axis of the pinhole camera model with the focal plane.

The distance formula for dImage between Point 1 and Point 2 in the image plane (which corresponds to the diametrical width of the tool in the image plane) may be expressed by:

(u2−u1)\hat{ }2+(v2−v1)\hat{ }2=dImage\hat{ }2 (5)

Substituting equations (1)-(4) for the values u1, v1, u2, and v2, respectively, and simplifying to isolate Z:

f\hat{ }2*(X2−X1)\hat{ }2/Z\hat{ }2+f\hat{ }2*(Y2−Y1)\hat{ }2/Z\hat{ }2=dImage\hat{ }2 (6)

f\hat{ }2*((X2−X1)\hat{ }2+(Y2−Y1)\hat{ }2))/Z\hat{ }2=dImage\hat{ }2 (7)

Z\hat{ }2=f\hat{ }2*((X2−X1)\hat{ }2+(Y2−Y1)\hat{ }2))/dImage\hat{ }2 (8)

For the physical instrument, the distance formula for the physical instrument width ToolWidth can be expressed with reference to the points that would correspond to image Points 1 and 2:

(X2−X1)\hat{ }2+(Y2−Y1)\hat{ }2=ToolWidth\hat{ }2 (9)

Substituting Z from Equation 8 into Equation 9 expresses the depth coordinate Z of the physical instrument tip as a function of the f parameter, the known image width at the tip (dImage) and the instrument's physical width (ToolWidth).

Z=f*ToolWidth/dImage (10)

With equation (10), the depth of the instrument's tip can thus be estimated from the known image width at the tip, the instrument's physical width, and the camera's f parameter.

Referring to Step 210, after estimating the depth Z, the X and Y of the tool's tip is estimated by:

X=Z*(uTip−px)/f (11)

Y=Z*(vTip−py)/f (12)

The tip in the image plane is the location that is identified by a small circle for each instrument in FIGS. 4 and 5, and that has coordinates uTip, vTip in the image plane.

The process continues until the X, Y, Z coordinates are estimated for the tip of each surgical instrument to be used for distance measurements. Once the X, Y and Z coordinates are estimated for the tip of each of the relevant surgical instruments, the distance between the tips can be estimated by calculating the Euclidean distance between their 3D coordinates (FIG. 2, Step 104) and communicated to the user (FIG. 2, Step 106). In FIGS. 4 and 5, the small circles identify the points at the distal end of each instrument between which the distance estimates are made. In FIG. 4, the distance estimate between the tips is displayed as a textual overlay. A graphical overlay in the form of a line between the instrument tips may also be displayed as shown in FIG. 4, showing the line along which the measurement is taken.

The system may continuously display the distance between the instrument tips, or it may do so as requested by the user. For example, when the instrument tips are at the desired measurement points, the user may give input instructing the system to display the measurement between the instrument tips. Types of input that can be used for this purpose are discussed below. The system may also be configured to retain in its memory one or more frames of the camera image as annotated with the measurement information (which may include one or more of the measured distance and units, graphics marking the measurement points, the line connecting the measurement points, or other data). This capture of the image and measurement data may occur automatically each time a measurement is taken, or in response to a command from the user using one of the user inputs. This aspect may be used in conjunction with aspects of the digital annotations described in co-pending and commonly owned U.S. application Ser. No. 17/679,080, which is incorporated herein by reference.

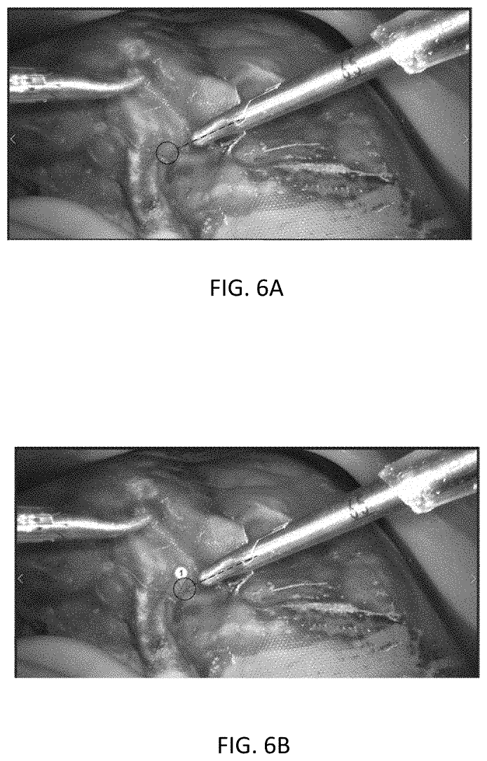

In slightly modified embodiments, measurements may be taken from points offset from the instrument tip such as described in U.S. application Ser. No. 17/099,761. For example, FIG. 6A shows a graphical indicator that is offset from the instrument on the display. This particular indicator has a circular border and a transparent interior, but other shapes and features could be used. In this embodiment, the surgical instrument is maneuvered at the surgical site to position the graphical indicator at a location (measurement point) from or at which the user wishes to take a measurement. Because the system can determine the X, Y and Z coordinates of the instrument tip, the X, Y and Z coordinates of an icon disposed on the longitudinal axis of the instrument and spaced a known distance from the instrument tip may be readily calculated by the system. As the user maneuvers the instruments robotically or manually at the surgical site, the graphical indicator moves with the associated instrument as if they are fixed or tethered to one another. Once the icons are at the desired point, the user gives input instructing the system to measure the distance between the two measurement points. In a specific embodiment, where the instrument is a robotically manipulated one, the user may operate an input device on the user input handle to signal to the system that the location at which the graphical indicator is located is to be received as a measurement point.

In some embodiments, the system may optionally give visual confirmation of a measurement point, whether it be at the instrument tip or at an offset marker, by dropping a graphical tag at the point, as shown in FIG. 6B. Subsequent measurement points may be optionally identified in a similar manner, and then the system may be instructed to display measurements between the identified measurement points or combinations of the points. Alternatively, as instruments are moved within the surgical site, measurements between the dropped tag and the tips of instruments within the field (e.g. as marked in FIG. 4, or 6A, or in other ways) may be displayed in real time as the instruments are moved.

In some embodiments, the system includes a device 16a by which the user gives input to the system that causes the system to set a measurement point and/or initiate a measurement between the identified measurement points at the instrument tips. As one example, the user may use the same input device 16 or type of input device described above. If a user input for a robotic system is used, the input device 16a might include a switch, button, touchpad, trackpad on the user input handle manipulated by the user to cause the manipulators to move the surgical instruments. Other inputs for use in robotic or non-robotic contexts include voice input devices, icons the user touches on a touch screen, foot pedal input, keyboard input, signals from a wireless input device mounted to the surgical instrument or the user's hand and activated by the user, signals from a switch pressed or moved by a body part such as a user's foot or knee, etc.

As discussed, in some embodiment the reference measurement may be the width of a particular portion of the tip instead of the width of the instrument's main shaft. In those alternative embodiments, the step of estimating the tool width in the image field (described in connection with FIG. 3, Step 206) would be conducted for that same portion of the tip.

The described system and method provide a number of advantages over prior art measurement techniques. Using the disclosed system, the method can be performed without first conducting a full camera calibration, since the only camera parameter needed is the f parameter value of a pinhole projective camera. Moreover, the method can be practiced using monocular cameras, allowing distance estimates to be obtained even where the system lacks stereoscopic camera capabilities.

It should also be noted that the instrument width can be used for other purposes other than estimating measurements. As an example, the processor may be programmed to display an overlay in the form of a marker on the instrument tip (e.g., where the circles are displayed on FIG. 4), and to cause the size of the displayed marker to change proportionally to the change in dImage as the surgical instrument moves in the field of view of the camera. This provides a 3D behavior to the marker as it changes its size as if it was a 3D object at the tool's tip location, giving the user visual input pertaining to the instrument position at the surgical site. Another example is text which is related to the instrument. The processor can be programmed to display and change the size and position of the text according to the instrument's tip location and width.

Robotic Surgical System

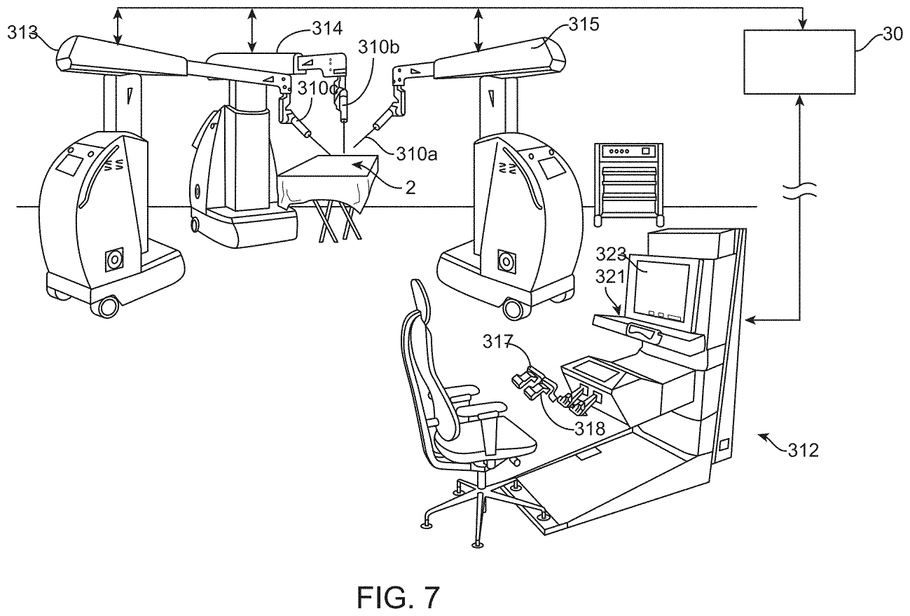

Although the concepts described herein may be used on a variety of robotic surgical systems, one robotic surgical system is shown in FIG. 7. In the illustrated system, a surgeon console 312 has two input devices such as handles 317, 318. These input devices are configured to be manipulated by a user to generate signals that are used to command motion of a robotically controlled device in multiple degrees of freedom. In use, the user selectively assigns the two handles 317, 318 to two of the robotic manipulators 313, 314, 315, allowing surgeon control of two of the surgical instruments 310a, 310b, and 310c disposed at the working site (in a patient on patient bed 2) at any given time. To control a third one of the instruments disposed at the working site, one of the two handles 317, 318 may be operatively disengaged from one of the initial two instruments and then operatively paired with the third instrument, or another form of input may control the third instrument as described in the next paragraph. A fourth robotic manipulator, not shown in FIG. 7, may be optionally provided to support and maneuver an additional instrument.

One of the instruments 310a, 310b, 310c is a camera that captures images of the operative field in the body cavity. In preferred embodiments, it is image data from this camera that is used in the described methods. The camera may be moved by its corresponding robotic manipulator using input from a variety of types of input devices, including, without limitation, one of the handles 317, 318, additional controls on the console, a foot pedal, an eye tracker 321, voice controller, etc. The console may also include a display or monitor 323 (which can be the display 14 discussed above) configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 317, 318 are configured to be manipulated by a user to generate signals that are processed by the system to generate instructions used to command motion of the manipulators in order to move the instruments in multiple degrees of freedom and to, as appropriate, control operation of electromechanical actuators/motors that drive motion and/or actuation of the instrument end effectors.

The surgical system allows the operating room staff to remove and replace the surgical instruments 310a, b, c carried by the robotic manipulator, based on the surgical need. When an instrument exchange is necessary, surgical personnel remove an instrument from a manipulator arm and replace it with another.

All prior patents and patent applications referred to herein, including for purposes of priority, are incorporated herein by reference.

We claim:

1. A system, comprising:
a camera positionable to capture image data corresponding to a treatment site;
at least one processor and at least one memory, the at least one memory storing instructions executable by said at least one processor to:
analyze image data to estimate a width in pixels of a first surgical instrument positioned at the treatment site;
estimate a depth coordinate Z of a tip of the first surgical instrument by comparing the estimated tool width to the known physical width of the first surgical instrument.

2. The system according to claim 1, wherein the instructions are further executable by said at least one processor to estimate X and Y coordinates of the first surgical instrument tip based on the Z coordinate.

3. The system according to claim 2, wherein the instructions are further executable by said at least one processor to:
analyze image data to estimate a width in pixels of a second surgical instrument positioned at the treatment site;
estimate a depth coordinate Z of a tip of the second surgical instrument by comparing the estimated second surgical instrument tool width to the known physical width of the second surgical instrument; and
estimate X and Y coordinates of the second surgical instrument tip based on the estimated Z coordinate of the second surgical instrument tip, and
estimate a distance between the first surgical instrument tip and the second instrument tip using the estimated X, Y and Z coordinates for the first instrument tip and the second instrument tip.

4. The system of claim 1, wherein the camera is a monocular camera.

5. The system of claim 1, wherein the camera is a stereoscopic camera.

6. The system of claim 1, wherein the only known intrinsic parameter of the camera is the f parameter.

7. A method comprising:
capturing image data of a treatment site using a camera positioned in a body cavity;
analyzing the image data to estimate a width in pixels of a first surgical instrument positioned at the treatment site;
estimating a depth coordinate Z of a tip of the first surgical instrument by comparing the estimated tool width to the known physical width of the first surgical instrument.

8. The method of claim 8, further comprising estimating X and Y coordinates of the first surgical instrument tip based on the Z coordinate.

9. The method according to claim 8, further including:
analyzing the image data to estimate a width in pixels of a second surgical instrument positioned at the treatment site;
estimating a depth coordinate Z of a tip of the second surgical instrument by comparing the estimated second surgical instrument tool width to the known physical width of the second surgical instrument;
estimating X and Y coordinates of the second surgical instrument tip based on the estimated Z coordinate of the second surgical instrument tip, and
estimating a distance between the first surgical instrument tip and the second instrument tip using the estimated X, Y and Z coordinates for the first instrument tip and the second instrument tip.

10. The method according to claim 9, further including displaying images from the camera on an image display, and displaying an overlay on the image display showing the distance estimated.

11. The method according to claim 10, further including displaying an overlay on the image identifying points between which the distance is measured.
